(19) United States
(12) Patent Application Publication
(10) Pub. No.: US 2023/0085573 A1
(43) Pub. Date: Mar. 16, 2023

(54) Compositions and Methods for Treating Aging-Related Disorders

(71) Applicant: AgeLess Sciences, Inc., New York, NY (US)

(72) Inventors: Kris Verburgh, Willebroek (BE); Chris Mirabile, West Palm Beach, FL (US)

(21) Appl. No.: 17/795,294

(22) PCT Filed: Feb. 1, 2021
(86) PCT No.: PCT/US2021/016003

§ 371 Date: Jul. 26, 2022

Related U.S. Application Data
(60) Provisional application No. 62/969,041, filed on Feb. 1, 2020.

(51) Int. Cl. A61K 31/194; A61K 31/09; A61K 36/9068; A61K 36/41; A61K 31/375; A61K 31/198; A61K 31/728; A61K 31/4412; A61K 31/522; A61K 31/132; A61K 33/00; A61K 31/352; A61K 31/7008; A61K 31/047; A61K 33/06; A61K 31/704; A61K 31/706

(52) U.S. Cl. CPC A61K 31/194; A61K 31/09; A61K 36/9068; A61K 36/41; A61K 31/375; A61K 31/198; A61K 31/728; A61K 31/4412; A61K 31/522; A61K 31/132; A61K 33/00; A61K 31/352; A61K 31/7008; A61K 31/047; A61K 33/06; A61K 31/704; A61K 31/706

(57) ABSTRACT

This invention provides a composition comprising at least three agents selected from the group consisting of alpha-ketoglutarate, malate, pterostilbene, micro-dosed lithium, glycine, ginger, Rhodiola rosea, acetyl glucosamine, vitamin C, glucosamine, fisetin, L-theanine, oxaloacetate, fumarate, succinate, hyaluronic acid, butyrate, anthocyanins, piperlongumine, quercetin, curcuminoids, caffeine, trehalose, and spermidine. This invention also provides related pharmaceutical compositions, methods for ameliorating hallmarks of aging, and articles of manufacture.

This application claims the benefit of U.S. Provisional Application No. 62/969,041, filed Feb. 1, 2020, the contents of which are incorporated herein by reference.

Throughout this application, various publications are cited. The disclosure of these publications is hereby incorporated by reference into this application to describe more fully the state of the art to which this invention pertains.

### FIELD OF THE INVENTION

The present invention relates to compositions and methods for ameliorating an array of age-related disorders.

### BACKGROUND OF THE INVENTION

Aging can be considered as a continuous decline of fitness and an intrinsic loss-of-function of the organism. This results in an increased risk of aging-related disease, dysfunction, and mortality.

Currently, many nutraceuticals and supplement formulations are based on outdated ideas about aging, such as Denham Harman's Free Radical Theory of Aging (1). That theory postulates that aging is mainly caused by free radicals that damage and impair cellular homeostasis. According to this dated theory, antioxidants (e.g., vitamin E, vitamin A, and coenzyme Q10) have the ability to neutralize free radicals, thus slowing the aging process. Yet, research shows that most antioxidants do not slow down aging, and could even shorten lifespan or undo the beneficial effects of exercise (2-6). For these and many other reasons, there is an unmet need for a superior oral formulation for ameliorating the hallmarks of aging.

### SUMMARY OF THE INVENTION

This invention provides a composition comprising at least three agents selected from the group consisting of alpha-ketoglutarate, malate, pterostilbene, micro-dosed lithium, glycine, ginger (preferably ginger extract), Rhodiola rosea, acetyl glucosamine, vitamin C, glucosamine, fisetin, L-theanine, oxaloacetate, fumarate, succinate, hyaluronic acid, butyrate, anthocyanins, piperlongumine, quercetin, curcuminoids (preferably curcumin), caffeine, trehalose, and spermidine.

This invention also provides a composition comprising (i) the present composition and (ii) a pharmaceutically acceptable carrier.

This invention further provides a method for ameliorating at least one aging hallmark in a subject comprising administering to the subject the present composition, wherein the aging hallmark is selected from the group consisting of genomic instability, telomere attrition, an epigenetic alteration, loss of proteostasis, deregulated nutrient sensing, mitochondrial dysfunction, cellular senescence, stem cell exhaustion, and altered intercellular communication.

Finally, this invention provides an article of manufacture comprising, in separate compartments, (i) the present composition and (ii) a nicotinamide-based compound (preferably nicotinamide mononucleotide).

### DETAILED DESCRIPTION OF THE INVENTION

This invention provides compositions and methods for ameliorating an array of age-related disorders.

### Definitions

In this application, certain terms are used which shall have the meanings set forth as follows.

As used herein, “administer”, with respect to an agent, means to deliver the agent to a subject's body via any known method. Specific modes of administration include, without limitation, intravenous, intramuscular, oral, sublingual, transdermal, subcutaneous, intraperitoneal, and intrathecal administration. Preferably, the present compositions are administered orally.

In addition, in this invention, the various compositions can be formulated using one or more routinely used pharmaceutically acceptable carriers. Such carriers are well known to those skilled in the art. For example, oral delivery systems include, for example, tablets, capsules, and powders (such as powders that can be added to water to form a drinkable formulation). These can contain excipients such as binders (e.g., hydroxypropylmethyl-cellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). In one embodiment, the oral delivery system is a powder and comprises one or more of xylitol, malic acid, flavoring, silicon dioxide, calcium silicate, and rebaudioside A.

As used herein, the term “aging hallmark” (used synonymously with “age-related hallmark” and “hallmark related to aging”) includes, without limitation, any of genomic instability, telomere attrition, an epigenetic alteration, loss of proteostasis, deregulated nutrient sensing, mitochondrial dysfunction, cellular senescence, stem cell exhaustion, and altered intercellular communication. These hallmarks are known, and reviewed in depth by C. López Otin, et al. (7).

As used herein, the term “ameliorate”, when used in reference to an aging hallmark, includes, without limitation, (i) slowing, stopping, reversing, or preventing the hallmark's progression, (ii) slowing, stopping, reversing, or preventing the progression of the hallmark's symptoms, (iii) preventing or reducing the likelihood of the hallmark's recurrence, and/or (iv) preventing or reducing the likelihood that the hallmark's symptoms will recur. In one embodiment, treating a subject afflicted with an aging hallmark means (i) reversing the hallmark's progression, ideally to the point of eliminating the hallmark, and/or (ii) reversing the progression of the hallmark's symptoms, ideally to the point of eliminating the symptoms.

As used herein, the term “subject” includes, without limitation, a mammal such as a human, a non-human primate, a dog, a cat, a horse, a sheep, a goat, a cow, a rabbit, a pig, a rat, and a mouse. Where the subject is human, the subject can be of any age. For example, the subject can be 60 years or older, 65 or older, 70 or older, 75 or older, 80 or older, 85 or older, or 90 or older. Alternatively, the subject can be 50 years or younger, 45 or younger, 40 or younger, 35 or younger, 30 or younger, 25 or younger, or 20 or younger.

### Embodiments of the Invention

This invention provides compositions and methods for ameliorating an array of age-related disorders. These compositions comprise certain combinations of agents that act in concert to address hallmarks of aging such as genomic instability, telomere attrition, epigenetic alterations, loss of proteostasis, deregulated nutrient sensing, mitochondrial dysfunction, cellular senescence, stem cell exhaustion, and altered intercellular communication. The invention also provides related articles of manufacture.

Specifically, this invention provides a composition comprising at least three agents selected from the group consisting of alpha-ketoglutarate, malate, pterostilbene, micro-dosed lithium, glycine, ginger (preferably ginger extract), Rhodiola rosea, acetyl glucosamine, vitamin C, glucosamine, fisetin, L-theanine, oxaloacetate, fumarate, succinate, hyaluronic acid, butyrate, anthocyanins, piperlongumine, quercetin, curcuminoids (preferably curcumin), caffeine, trehalose, and spermidine. In one embodiment, the composition comprises at least three agents selected from the group consisting of alpha-ketoglutarate, malate, pterostilbene, micro-dosed lithium, glycine, ginger extract, Rhodiola rosea, acetyl glucosamine, vitamin C, glucosamine, fisetin, and L-theanine.

Each of these agents is commercially available in oral dosage forms. The following are non-limiting examples. Alpha-ketoglutarate, also known as alpha-ketoglutaric acid, is commercially available, for example, in 1,000 mg capsules. Alpha-ketoglutarate is available as the calcium salt, but all other salts of alpha-ketoglutarate are envisioned in this invention. Malate, also known as malic acid, is commercially available, for example, as magnesium malate in 500 mg capsules. Also envisioned for use in this invention, in lieu of malate, are fumarate and oxaloacetate. Pterostilbene is commercially available, for example, in 100 mg capsules. Micro-dosed lithium, also known as microdose lithium, is commercially available, for example, in 1,000 mcg capsules. Glycine is commercially available, for example, in 1,000 mg capsules. Ginger is commercially available as ginger root and as ginger extract (preferably organic ginger extract, standardized to 2% gingerols, for example, PurGinger™ (Applied Food Sciences)). Rhodiola rosea, preferably with a minimum of 3% rosavins and 2% salidrosides, is commercially available, for example, in 300 mg capsules. Acetyl-glucosamine, also known as N-acetylglucosamine, is commercially available, for example, in 600 mg capsules. Glucosamine can be used by itself, in its salt form (e.g., as glucosamine sulphate, glucosamine HCl, glucosamine 2KCI), and in its acylated form (i.e., acetyl-glucosamine). Acetyl-glucosamine is also a component of hyaluronic acid (one type of acetyl-glucosamine-containing polysaccharide). As such, acetyl-glucosamine can also be delivered to a subject in the form of hyaluronic acid. Vitamin C, also known as ascorbic acid and ascorbate, is commercially available, for example, in 1,000 mg tablets. Glucosamine is commercially available, for example, as glucosamine sulfate, in capsules containing 400 mg of glucosamine sulfate. Fisetin is commercially available, for example, in 100 mg capsules. L-theanine, also known as L-γ-glutamylethylamide and N⁵-ethyl-L-glutamine, is commercially available, for example, in 100 mg capsules. Spermidine is commercially available, for example, in 400 mg capsules of wheat germ extract containing 0.5 mg of spermidine. Finally, trehalose is commercially available in powder form.

Envisioned for the present composition are various combinations of agents. In one embodiment, the present composition comprises alpha-ketoglutarate, micro-dosed lithium, and fisetin. In another embodiment, the present composition comprises alpha-ketoglutarate, fisetin, pterostilbene, and glycine. In another embodiment, the present composition comprises alpha-ketoglutarate, micro-dosed lithium, and pterostilbene. In another embodiment, the present composition comprises alpha-ketoglutarate, micro-dosed lithium, and glycine. In another embodiment, the present composition comprises alpha-ketoglutarate, pterostilbene, and fisetin. In another embodiment, the present composition comprises pterostilbene, micro-dosed lithium, and fisetin. In another embodiment, the present composition comprises alpha-ketoglutarate, micro-dosed lithium, fisetin, glycine, and pterostilbene. In another embodiment, the present composition comprises alpha-ketoglutarate, micro-dosed lithium, fisetin, glycine, and vitamin C. In a further embodiment, the present composition comprises alpha-ketoglutarate, pterostilbene, micro-dosed lithium, glycine, glucosamine, and fisetin. Additional embodiments are set forth below in the Examples section.

In yet a further embodiment, the present composition comprises alpha-ketoglutarate, malate, pterostilbene, micro-dosed lithium, glycine, ginger (preferably ginger extract), Rhodiola rosea, hyaluronic acid, vitamin C, glucosamine, fisetin, and L-theanine. Preferably, the present composition comprises 1,100 mg of calcium alpha-ketoglutarate, 2,000 mg of magnesium malate, 50 mg of pterostilbene, 1 mg of micro-dosed lithium, 2,000 mg of glycine, 80 mg of ginger extract, 300 mg of Rhodiola rosea, 100 mg of hyaluronic acid, 100 mg of vitamin C, 1,000 mg of glucosamine sulphate, 100 mg of fisetin, and 150 mg of L-theanine. In still a further embodiment, the present composition comprises 1,100 mg of calcium alpha-ketoglutarate, 2,000 mg of magnesium malate, 50 mg of pterostilbene, 1 mg of micro-dosed lithium, 2,000 mg of glycine, 80 mg of ginger extract, 300 mg of Rhodiola rosea, 100 mg of hyaluronic acid, 100 mg of vitamin C, 1,000 mg of glucosamine sulphate, 100 mg of fisetin, 200 mg of L-theanine, xylitol, malic acid, flavoring, silicon dioxide, calcium silicate, and rebaudioside A. Optionally, the present composition comprises one or more sweeteners such as, without limitation, erythritol, trehalose, maltitol, mannitol, sorbitol, xylitol, isomalt, lactitol, tagatose, monk fruit, inuline, and stevia-derived substances. Preferably, the present composition (e.g., the embodiments described in this paragraph) is in the form of a powder that, when admixed with a fluid such as water, can be taken orally as a drink by the subject (preferably once daily).

Preferably, one or more of the agents in the present composition is formulated for delayed release and/or extended release. In one embodiment of the present composition, the alpha-ketoglutarate, micro-dosed lithium, fisetin, butyrate, pterostilbene, and glycine are formulated for delayed release and/or extended release. In another embodiment of the present composition, each agent is formulated for delayed release and/or extended release. In still a further embodiment, the present composition comprises 1,100 mg of delayed release and/or extended release calcium alpha-ketoglutarate, 2,000 mg of magnesium malate, 50 mg of delayed release and/or extended release pterostilbene, 1 mg of delayed release and/or extended release micro-dosed lithium, 2,000 mg of delayed release and/or extended release glycine, 80 mg of ginger extract, 300 mg of Rhodiola rosea, 100 mg of hyaluronic acid, 100 mg of vitamin C, 1,000 mg of glucosamine sulphate, 100 mg of delayed release and/or extended release fisetin, 150 mg of L-theanine, xylitol, malic acid, flavoring, silicon dioxide, calcium silicate, and rebaudioside A. Optionally, the present composition comprises one or more sweeteners such as, without limitation, erythritol, trehalose, maltitol, mannitol, sorbitol, xylitol, isomalt, lactitol, tagatose, monk fruit, inuline, or stevia-derived substances.

This invention also provides a composition comprising (i) the present composition and (ii) a pharmaceutically acceptable carrier.

This invention further provides a method for ameliorating at least one aging hallmark in a subject comprising administering to the subject the present composition (preferably once daily), wherein the aging hallmark is selected from the group consisting of genomic instability, telomere attrition, an epigenetic alteration, loss of proteostasis, deregulated nutrient sensing, mitochondrial dysfunction, cellular senescence, stem cell exhaustion, and altered intercellular communication. In one embodiment, the present method ameliorates genomic instability. In another embodiment, the present method ameliorates telomere attrition. In another embodiment, the present method ameliorates one or more epigenetic alterations. In another embodiment, the present method ameliorates loss of proteostasis. In another embodiment, the present method ameliorates deregulated nutrient sensing. In another embodiment, the present method ameliorates mitochondrial dysfunction. In another embodiment, the present method ameliorates cellular senescence. In another embodiment, the present method ameliorates stem cell exhaustion. In another embodiment, the present method ameliorates altered intercellular communication.

In a further embodiment of the present method, the method ameliorates at least two aging hallmarks in the subject. In another embodiment, the present method ameliorates at least three aging hallmarks in the subject. In another embodiment, the present method ameliorates at least four aging hallmarks in the subject. In another embodiment, the present method ameliorates at least five aging hallmarks in the subject. In another embodiment, the present method ameliorates at least six aging hallmarks in the subject. In another embodiment, the present method ameliorates at least seven aging hallmarks in the subject. In another embodiment, the present method ameliorates at least eight aging hallmarks in the subject. In another embodiment, the present method ameliorates at least nine aging hallmarks in the subject.

For example, in one embodiment, the present method ameliorates at least the following aging hallmarks in the subject: genomic instability, an epigenetic alteration, and loss of proteostasis. In another embodiment, the present method ameliorates at least the following aging hallmarks in the subject: an epigenetic alteration, loss of proteostasis, and mitochondrial dysfunction. In another embodiment, the present method ameliorates at least the following aging hallmarks in the subject: an epigenetic alteration, loss of proteostasis, and deregulated nutrient sensing. In another embodiment, the present method ameliorates cellular senescence or reduces the amount of senescent cells. In another embodiment, the present method ameliorates cellular senescence and stem cell exhaustion. In another embodiment, the present method ameliorates mitochondrial dysfunction and loss of proteostasis. In another embodiment, the present method ameliorates epigenetic alterations and loss of proteostasis. In another embodiment, the present method ameliorates altered intercellular communication and deregulated nutrient sensing. In another embodiment, the present method ameliorates altered intercellular communication and stem cell exhaustion. In another embodiment, the present method ameliorates stem cell exhaustion and epigenetic alterations. In another embodiment, the present method ameliorates epigenetic alterations and mitochondrial dysfunction. In another embodiment, the present method ameliorates epigenetic alterations, loss of proteostasis, and mitochondrial dysfunction. In another embodiment, the present method ameliorates stem cell exhaustion, cellular senescence, and altered intercellular communication. In another embodiment, the present method ameliorates epigenetic alterations, cellular senescence, and mitochondrial dysfunction. In another embodiment, the present method ameliorates deregulated nutrient sensing, mitochondrial dysfunction, and loss or proteostasis. In another embodiment, the present method ameliorates epigenetic alterations, loss of proteostasis, mitochondrial dysfunction, and cellular senescence.

Preferably, the present method further comprises concurrently administering a nicotinamide-based compound (preferably nicotinamide mononucleotide) to the subject. In this invention, “concurrently administering” a nicotinamide-based compound and the present composition includes, for example, administering the nicotinamide-based compound (i) at the same time as, or (ii) within one hour of, within three hours of, within six hours of, within 12 hours of, or within 24 hours of, the present composition. Nicotinamide-based compounds include, without limitation, nicotinamide adenine dinucleotide (NAD), nicotinamide mononucleotide, nicotinic acid (i.e., niacin), nicotinamide (NAM), nicotinamide riboside (NR), nicotinic acid riboside (NaR), and nicotinic acid mononucleotide (NaNM). In a preferred embodiment, the present method further comprises concurrently administering 125 mg of nicotinamide mononucleotide to the subject in capsule form. Nicotinamide mononucleotide (also known as NMN, nicotinamide ribonucleoside 5-phosphate, nicotinamide D-ribonucleotide, β-Nicotinamide ribose monophosphate, and nicotinamide nucleotide) is commercially available in capsule forms of various dosages (e.g., 100 mg and 300 mg).

In this invention, it is envisioned that the present method ameliorates genomic instability, telomere attrition, one or more epigenetic alterations, loss of proteostasis, deregulated nutrient sensing, mitochondrial dysfunction, cellular senescence, stem cell exhaustion, and altered intercellular communication.

Preferably, in the present method, the subject is a human. In another embodiment, the subject is a cat, a dog, or a horse.

Finally, this invention provides an article of manufacture comprising, in separate compartments, (i) the present composition and (ii) a nicotinamide-based compound (preferably nicotinamide mononucleotide). In one embodiment, the present article comprises, in separate compartments, (i) a composition comprising alpha-ketoglutarate, malate, pterostilbene, micro-dosed lithium, glycine, ginger (preferably ginger extract), Rhodiola rosea, hyaluronic acid, vitamin C, glucosamine, fisetin, and L-theanine, and (ii) a nicotinamide-based compound (preferably nicotinamide mononucleotide).

Preferably, the present article comprises, in separate compartments, (i) a composition (preferably in powder form) comprising 1,100 mg of calcium alpha-ketoglutarate, 2,000 mg of magnesium malate, 50 mg of pterostilbene, 1 mg of micro-dosed lithium, 2,000 mg of glycine, 80 mg of ginger extract, 300 mg of Rhodiola rosea, 100 mg of hyaluronic acid, 100 mg of vitamin C, 1,000 mg of glucosamine sulphate, 100 mg of fisetin, and 150 mg of L-theanine (and optionally also xylitol, malic acid, flavoring, silicon dioxide, calcium silicate, and rebaudioside A), and (ii) 125 mg of nicotinamide mononucleotide (preferably in capsule form).

In the present method, it is envisioned that the present composition is orally administered to the subject daily, preferably concurrently with a nicotinamide-based compound (preferably nicotinamide mononucleotide). The following dosages are envisioned, without limitation: (i) 1,100 mg of calcium alpha-ketoglutarate (or optionally, any of 100 mg, 200 mg, 300 mg, 400 mg, 500 mg, 600 mg, 700 mg, 800 mg, 900 mg, 1,000 mg, 1,200 mg, 1,300 mg, 1,400 mg, 1,500 mg, 1,600 mg, 1,700 mg, 1,800 mg, 1,900 mg, or 2,000 mg); (ii) 2,000 mg of magnesium malate (or optionally, any of 200 mg, 400 mg, 600 mg, 800 mg, 1,000 mg, 1,200 mg, 1,400 mg, 1,600 mg, 1,800 mg, 2,200 mg, 2,400 mg, 2,600 mg, 2,800 mg, 3,000 mg, 3,200 mg, 3,400 mg, 3,600 mg, 3,800 mg, or 4,000 mg); (iii) 50 mg of pterostilbene (or optionally, any of 10 mg, 20 mg, 30 mg, 40 mg, 60 mg, 70 mg, 80 mg, 90 mg, or 100 mg); (iv) 1 mg of micro-dosed lithium (or optionally, any of 0.1 mg, 0.2 mg, 0.3 mg, 0.4 mg, 0.5 mg, 0.6 mg, 0.7 mg, 0.8 mg, 0.9 mg, 1.1 mg, 1.2 mg, 1.3 mg, 1.4 mg, 1.5 mg, 1.6 mg, 1.7 mg, 1.8 mg, 1.9 mg, or 2.0 mg); (v) 2,000 mg of glycine (or optionally, any of 200 mg, 400 mg, 600 mg, 800 mg, 1,000 mg, 1,200 mg, 1,400 mg, 1,600 mg, 1,800 mg, 2,200 mg, 2,400 mg, 2,600 mg, 2,800 mg, 3,000 mg, 3,200 mg, 3,400 mg, 3,600 mg, 3,800 mg, or 4,000 mg); (vi) 80 mg of ginger extract (or optionally, any of 10 mg, 20 mg, 30 mg, 40 mg, 60 mg, 70 mg, 90 mg, 100 mg, 110 mg, 120 mg, 130 mg, 140 mg, 150 mg, 160 mg, 170 mg, 180 mg, 190 mg, 200 mg, 250 mg, 300 mg, 350 mg, 400 mg, 450 mg, 500 mg, 600 mg, 700 mg, 800 mg, 900 mg, or 1,000 mg); (vii) 300 mg of Rhodiola rosea (or optionally, any of 100 mg, 150 mg, 200 mg, 250 mg, 350 mg, 400 mg, 450 mg, or 500 mg); (viii) 100 mg of hyaluronic acid (or optionally, any of 10 mg, 20 mg, 30 mg, 40 mg, 60 mg, 70 mg, 80 mg, 90 mg, 110 mg, 120 mg, 130 mg, 140 mg, 150 mg, 160 mg, 170 mg, 180 mg, 190 mg, or 200 mg); (ix) 100 mg of vitamin C (or optionally, any of 10 mg, 20 mg, 30 mg, 40 mg, 60 mg, 70 mg, 80 mg, 90 mg, 110 mg, 120 mg, 130 mg, 140 mg, 150 mg, 160 mg, 170 mg, 180 mg, 190 mg, or 200 mg); (x) 1,000 mg of glucosamine sulphate (or optionally, any of 200 mg, 400 mg, 600 mg, 800 mg, 1,200 mg, 1,400 mg, 1,600 mg, 1,800 mg, or 2,000 mg); (xi) 100 mg of fisetin (or optionally, any of 10 mg, 20 mg, 30 mg, 40 mg, 60 mg, 70 mg, 80 mg, 90 mg, 110 mg, 120 mg, 130 mg, 140 mg, 150 mg, 160 mg, 170 mg, 180 mg, 190 mg, 200 mg, 250 mg, 300 mg, 350 mg, 400 mg, 450 mg, 500 mg, 600 mg, 700 mg, 800 mg, 900 mg, 1,000 mg, 1,100 mg, 1,200 mg, 1,300 mg, 1,400 mg, 1,500 mg, 1,600 mg, 1,700 mg, 1,800 mg, 1,900 mg, or 2,000 mg); (xii) 150 mg of L-theanine (or optionally, any of 20 mg, 40 mg, 60 mg, 80 mg, 100 mg, 120 mg, 140 mg, 160 mg, 180 mg, 200 mg, 220 mg, 240 mg, 260 mg, 280 mg, or 300 mg); (xiii) 3 mg of spermidine (or optionally, any of 0.1 mg, 0.5 mg, 1 mg, 2 mg, 4 mg, 5 mg, 6 mg, 7 mg, 8 mg, 9 mg, 10 mg, 11 mg, or 12 mg); (xiv) 15 g of trehalose (or optionally, any of 1 g, 2 g, 5 g, 10 g, 20 g, 30 g, 40 g, or 50 g); and (xv) 125 mg of nicotinamide mononucleotide (or optionally, any of 10 mg, 20 mg, 30 mg, 40 mg, 50 mg, 60 mg, 70 mg, 80 mg, 90 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 300 mg, 350 mg, 400 mg, 450 mg, 500 mg, 600 mg, 700 mg, 800 mg, 900 mg, 1,000 mg, 1,100 mg, 1,200 mg, 1,300 mg, 1,400 mg, 1,500 mg, 1,600 mg, 1,700 mg, 1,800 mg, 1,900 mg, or 2,000 mg).

The following three dosage regimens are (i) also envisioned for daily oral administration (in delayed release and/or extended release form, as well as non-delayed release, non-extended release form) and (ii) envisioned for the present compositions and articles of manufacture. They comprise, without limitation, the following: (i) 275 mg of calcium alpha-ketoglutarate, 500 mg of magnesium malate, 12.5 mg of pterostilbene, 0.25 mg of micro-dosed lithium, 500 mg of glycine, 20 mg of ginger extract, 75 mg of Rhodiola rosea, 25 mg of hyaluronic acid, 25 mg of vitamin C, 250 mg of glucosamine sulphate, 25 mg of fisetin, and 37.5 mg of L-theanine; (ii) 550 mg of calcium alpha-ketoglutarate, 1,000 mg of magnesium malate, 25 mg of pterostilbene, 0.5 mg of micro-dosed lithium, 1,000 mg of glycine, 40 mg of ginger extract, 150 mg of Rhodiola rosea, 50 mg of hyaluronic acid, 50 mg of vitamin C, 500 mg of glucosamine sulphate, 50 mg of fisetin, and 75 mg of L-theanine; and (iii) 825 mg of calcium alpha-ketoglutarate, 1,500 mg of magnesium malate, 37.5 mg of pterostilbene, 0.75 mg of micro-dosed lithium, 1,500 mg of glycine, 60 mg of ginger extract, 225 mg of Rhodiola rosea, 75 mg of hyaluronic acid, 75 mg of vitamin C, 750 mg of glucosamine sulphate, 75 mg of fisetin, and 112.5 mg of L-theanine.

This invention will be better understood by reference to the examples which follow, but those skilled in the art will readily appreciate that the specific examples detailed are only illustrative of the invention as described more fully in the claims which follow thereafter.

### EXAMPLES

### Example 1—Additional Embodiments of the Present Compositions

The following additional combinations of agents are envisioned, without limitation, for the present compositions.

The first additional combination comprises alpha-ketoglutarate, micro-dosed lithium, fisetin, and one or more additional agents selected from malate, pterostilbene, glycine, ginger (preferably ginger extract), Rhodiola rosea, acetyl glucosamine, vitamin C, glucosamine, and L-theanine.

The second additional combination comprises alpha-ketoglutarate, micro-dosed lithium pterostilbene, and one or more additional agents selected from malate, glycine, ginger (preferably ginger extract), Rhodiola rosea, acetyl glucosamine, vitamin C, glucosamine, fisetin, and L-theanine.

The third additional combination comprises alpha-ketoglutarate, micro-dosed lithium, glycine, and one or more additional agents selected from malate, pterostilbene, ginger (preferably ginger extract), Rhodiola rosea, acetyl glucosamine, vitamin C, glucosamine, fisetin, and L-theanine.

The fourth additional combination comprises alpha-ketoglutarate, pterostilbene, fisetin, and one or more additional agents selected from malate, micro-dosed lithium, glycine, ginger (preferably ginger extract), Rhodiola rosea, acetyl glucosamine, vitamin C, glucosamine, and L-theanine.

The fifth additional combination comprises pterostilbene, micro-dosed lithium, fisetin, and one or more additional agents selected from alpha-ketoglutarate, malate, glycine, ginger (preferably ginger extract), Rhodiola rosea, acetyl glucosamine, vitamin C, glucosamine, and L-theanine.

The sixth additional combination comprises alpha-ketoglutarate, micro-dosed lithium, fisetin, glycine, vitamin C, and one or more additional agents selected from malate, pterostilbene, ginger (preferably ginger extract), Rhodiola rosea, acetyl glucosamine, glucosamine, and L-theanine.

The seventh additional combination comprises alpha-ketoglutarate, pterostilbene, micro-dosed lithium, glycine, glucosamine, fisetin, and one or more additional agents selected from malate, ginger (preferably ginger extract), Rhodiola rosea, vitamin C, glucosamine, and L-theanine.

The eighth additional combination comprises glucosamine, malate, glycine, lithium, pterostilbene, fisetin, hyaluronic acid, alpha-ketoglutarate, ginger (preferably ginger extract), anthocyanins, rhodiola, spermidine, vitamin C, or combinations thereof. 20 The ninth additional combination comprises glucosamine, malate, glycine, lithium, pterostilbene, fisetin, alpha-ketoglutarate, ginger (preferably ginger extract), anthocyanins, rhodiola, spermidine, vitamin C, or combinations thereof.

The tenth additional combination comprises glucosamine, malate, glycine, lithium, 25 pterostilbene, fisetin, alpha-ketoglutarate, ginger (preferably ginger extract), anthocyanins, spermidine, vitamin C, or combinations thereof.

The eleventh additional combination comprises glucosamine, malate, glycine, lithium, pterostilbene, fisetin, alpha-ketoglutarate, anthocyanins, spermidine, vitamin C, or 30 combinations thereof.

The twelfth additional combination comprises glucosamine, malate, glycine, lithium, pterostilbene, fisetin, alpha-ketoglutarate, anthocyanins, vitamin C, or combinations thereof.

The thirteenth additional combination comprises lithium, malate, and alpha-ketoglutarate, with or without vitamin C.

The fourteenth additional combination comprises lithium, glycine, malate, and alpha-ketoglutarate, with or without vitamin C.

The fifteenth additional combination comprises lithium, glycine, malate, fisetin, and alpha-ketoglutarate, with or without vitamin C.

The sixteenth additional combination comprises alpha-ketoglutarate, micro-dosed lithium, and glucosamine.

The seventeenth additional combination comprises alpha-ketoglutarate, micro-dosed lithium, fisetin, and pterostilbene.

The eighteenth additional combination comprises alpha-ketoglutarate, micro-dosed lithium, fisetin, and glucosamine.

The nineteenth additional combination comprises alpha-ketoglutarate, micro-dosed lithium, fisetin, and glycine.

The twentieth additional combination comprises alpha-ketoglutarate, micro-dosed lithium, fisetin, pterostilbene, and glucosamine.

The twenty-first additional combination comprises alpha-ketoglutarate, fisetin, and spermidine.

The twenty-second additional combination comprises alpha-ketoglutarate, fisetin, spermidine, and trehalose.

The twenty-third additional combination comprises alpha-ketoglutarate, fisetin, spermidine, and pterostilbene.

The twenty-fourth additional combination comprises alpha-ketoglutarate, fisetin, and trehalose.

The twenty-fifth additional combination comprises alpha-ketoglutarate, fisetin, trehalose, and pterostilbene.

The twenty-sixth additional combination comprises alpha-ketoglutarate, micro-dosed lithium, fisetin, and spermidine.

The twenty-seventh additional combination comprises alpha-ketoglutarate, micro-dosed lithium, fisetin, and trehalose.

The twenty-eighth additional combination comprises alpha-ketoglutarate, micro-dosed lithium, fisetin, spermidine, and trehalose.

The twenty-ninth additional combination comprises alpha-ketoglutarate, fisetin, and butyric acid.

The thirtieth additional combination comprises alpha-ketoglutarate, fisetin, pterostilbene, and butyric acid.

The thirty-first additional combination comprises alpha-ketoglutarate, micro-dosed lithium, fisetin, and butyric acid.

The thirty-second additional combination comprises malate, glycine, lithium, pterostilbene, fisetin, alpha-ketoglutarate, anthocyanins, vitamin C, or combinations thereof.

The thirty-third additional combination comprises fisetin, pterostilbene, and glucosamine.

The thirty-fourth additional combination comprises fisetin, glycine, and pterostilbene.

The thirty-fifth additional combination comprises fisetin, glucosamine, and glycine.

The thirty-sixth additional combination comprises pterostilbene, glycine, and glucosamine.

The thirty-seventh additional combination comprises fisetin, pterostilbene, glucosamine and glycine.

The thirty-eighth additional combination comprises fisetin, pterostilbene, glucosamine, glycine, and malate.

The thirty-ninth additional combination comprises fisetin, pterostilbene, and acetyl-glucosamine.

The fortieth additional combination comprises fisetin, pterostilbene, glucosamine, glycine, and acetyl-glucosamine.

The forty-first additional combination comprises fisetin, pterostilbene, and theanine.

The forty-second additional combination comprises alpha-ketoglutarate, fisetin, and theanine.

The forty-third additional combination comprises alpha-ketoglutarate, micro-dosed lithium, and theanine.

The forty-fourth additional combination comprises alpha-ketoglutarate, micro-dosed lithium, fisetin, and theanine.

The forty-fifth additional combination comprises alpha-ketoglutarate, micro-dosed lithium, fisetin, pterostilbene, and theanine.

The forty-sixth additional combination comprises alpha-ketoglutarate, micro-dosed lithium, fisetin, pterostilbene, glucosamine, and theanine.

For each of the above additional combinations, it is envisioned that a nicotinamide-based compound (preferably NMN) would be administered to the subject concurrently.

The following NMN-containing combinations of agents are also envisioned, without limitation, for the present compositions: (i) a composition comprising nicotinamide mononucleotide, alpha-ketoglutarate, and fisetin; (ii) a composition comprising nicotinamide mononucleotide, alpha-ketoglutarate, micro-dosed lithium, and fisetin; (iii) a composition comprising nicotinamide mononucleotide, alpha-ketoglutarate, pterostilbene, and fisetin; (iv) a composition comprising nicotinamide mononucleotide, pterostilbene, and fisetin; (v) a composition comprising nicotinamide mononucleotide, pterostilbene, fisetin and micro-dosed lithium; and (vi) a composition comprising nicotinamide mononucleotide, pterostilbene, fisetin, and glucosamine.

### Example 2—Measuring Epigenetic Dysregulation

The success of the present composition in ameliorating epigenetic dysregulation in a subject (e.g., a human subject) can be measured, for example, by making any of the following determinations: (i) the subject's epigenetic methylation, histonylation, and/or chromatin patterns more closely resemble the patterns of younger persons (e.g., by at least 1%, at least 2%, at least 5%, at least 10%, at least 20%, at least 30%, at least 40%, at least 50%, at least 60%, at least 70%, at least 80%, at least 90%, or at least 100%); (ii) an epigenetic clock (e.g., a Horvath clock or a Levine clock)-measuring methylation of DNA in different regions of the subject's DNA more closely resembles the DNA methylation pattern of a younger subject (e.g., by at least 1%, at least 2%, at least 5%, at least 10%, at least 20%, at least 30%, at least 40%, at least 50%, at least 60%, at least 70%, at least 80%, at least 90%, or at least 100%); (iii) an epigenetic DNA methylation clock (e.g., a Horvath clock or a Levine clock) shows that an adult human subject has been rejuvenated by at least one year, at least two years, or at least three years after being administered the present composition (or of which the epigenetic age or biological age has been decreased by at least 1%, at least 2%, at least 3%, at least 5%, at least 10%, at least 20%, at least 30%, at least 40%, at least 50%, at least 60%, at least 70%, at least 80%, at least 90%, or at least 100% compared to a subject's chronological age); (iv) the subject's chromatin distribution more closely resembles that of a younger subject (e.g., by at least 0.2%, at least 1%, at least 2%, at least 5%, at least 10%, at least 20%, at least 30%, at least 40%, at least 50%, at least 60%, at least 70%, at least 80%, at least 90%, or at least 100%); (v) the subject's transcriptome resembles the transcriptome of a younger subject (e.g., by at least 0.2%, at least 0.5%, at least 1%, at least 2%, at least 5%, at least 10%, at least 20%, at least 30%, at least 40%, at least 50%, at least 60%, at least 70%, at least 80%, at least 90%, or at least 100%); (vi) the subject's expression profile of ribosome-related proteins and/or ribosome-related RNA more closely resembles that of a younger organism (e.g., by at least 0.2%, at least 0.5%, at least 1%, at least 2%, at least 5%, at least 10%, at least 20%, at least 30%, at least 40%, at least 50%, at least 60%, at least 70%, at least 80%, at least 90%, or at least 100%); or (vii) the subject's levels of sirtuins have increased (e.g., by at least 1%, at least 2%, at least 5%, at least 10%, at least 20%, at least 30%, at least 40%, at least 50%, at least 60%, at least 70%, at least 80%, at least 90%, or at least 100%).

The success of the present composition in ameliorating epigenetic dysregulation in a subject (e.g., a human subject) can also be measured, for example, by making any of the following determinations: (i) an increase in the level of H3K9me3 in the subject (e.g., by at least 1%, at least 2%, at least 5%, at least 10%, at least 20%, at least 30%, at least 40%, at least 50%, at least 60%, at least 70%, at least 80%, at least 90%, or at least 100%); or (ii) an increase in the level of heterochromatin protein (Hp1y) in the subject (e.g., by at least 1%, at least 2%, at least 5%, at least 10%, at least 20%, at least 30%, at least 40%, at least 50%, at least 60%, at least 70%, at least 80%, at least 90%, or at least 100%).

### Example 3—Measuring Cell Senescence

The success of the present composition in ameliorating cell senescence in a subject (e.g., a human subject) can be measured, for example, by making any of the following determinations: (i) a delay in the occurrence of senescent cells or a delay in the transition of normal cells into senescent cells in vitro or in vivo (e.g., by at least 2%, at least 5%, at least 10%, at least 20%, at least 30%, at least 40%, at least 50%, at least 60%, at least 70%, at least 80%, at least 90%, or at least 100%); and/or (ii) a decrease in the number of senescent cells in vitro or in vivo (e.g., by at least 1%, at least 2%, at least 5%, at least 10%, at least 20%, at least 30%, at least 40%, at least 50%, at least 60%, at least 70%, at least 80%, at least 90%, or at least 100%). Senescence or senescent-like status of cells can be measured, for example, via one or more of the following: (i) epigenetic profiling such as measuring epigenetic markers of senescence (e.g., changes in histone H3 lysine 9 and 27 trimethylation (changed levels of H3K9me3 and H3K27me3), changed heterochromatin protein 1 (HP1) family protein levels, increased histone variant macroH2A levels, and/or chromatin remodeling enzyme ATRX levels); (ii) changes in cell morphology resembling the morphology of senescent cells, (e.g., enlarged size, a more flattened shape, polyploid nuclei, or accumulation of DNA damage foci); (iii) changes in levels of nuclear lamina-associated proteins such as lamins (e.g., a decline lamin B1); (iv) heterochromatin changes more resembling the heterochromatin status of senescent cells (e.g., an increase in senescence-associated heterochromatic foci (SAHF)); (v) an increase of senescence-associated distention of satellites (SADS) or other changes in pericentric satellite DNA more resembling that of senescent cells; and (vi) changes in secretory phenotype corresponding to that of senescent cells (e.g., the occurrence of a more senescence-associated secretome including, without limitation, cytokines, interleukins (e.g., IL-1, IL-2, IL-6, II-8, and TNF-alpha), matrix metalloproteinases, pro angiogenetic factors, pro-inflammatory substances, and growth factors (e.g., vascular endothelial growth factor, insulin like growth factor, and GM-CSF)).

The success of the present composition in ameliorating cell senescence in a subject (e.g., a human subject) can also be measured, for example, by making any of the following determinations: (i) an increase in the level of H3K9me3 in the subject (e.g., by at least 5%, at least 10%, at least 20%, at least 30%, at least 40%, at least 50%, at least 60%, at least 70%, at least 80%, at least 90%, or at least 100%); (ii) an increase in the level of H3K27me3 in the subject (e.g., by at least 5%, at least 10%, at least 20%, at least 30%, at least 40%, at least 50%, at least 60%, at least 70%, at least 80%, at least 90%, or at least 100%); (iii) a decrease in the level of P16INK4A in the subject (e.g., by at least 5%, at least 10%, at least 20%, at least 30%, at least 40%, at least 50%, at least 60%, at least 70%, at least 80%, at least 90%, or at least 100%); or (iv) a decrease in the level of one or more of p16, p53, p21, p14, p15, and beta-galactosidase activity in the subject (e.g., by at least 5%, at least 10%, at least 20%, at least 30%, at least 40%, at least 50%, at least 60%, at least 70%, at least 80%, at least 90%, or at least 100%).

### Example 4—Measuring Altered Intercellular Communication

The success of the present composition in ameliorating altered intercellular communication in a subject (e.g., a human subject) can be measured, for example, by determining a decrease in the level of one or more of IL-1, IL-2, IL-6, IL-8, IL-17a, and TNF-alpha in the subject (e.g., by at least 5%, at least 10%, at least 20%, at least 30%, at least 40%, at least 50%, at least 60%, at least 70%, at least 80%, at least 90%, or at least 100%).

### Example 5—Measuring Autophagy

The success of the present composition in ameliorating decreased autophagy in a subject (e.g., a human subject) can be measured, for example, by determining any of the following: (i) an increase in the level of one or more of LC3, p62, Ulk1, Parkin, PINK1, LAMP2A, Atg, FIP200, Vps15, Beclin, Lamp-1, Lamp-2, Hsp70, Hsp90, and SQSTM1 in the subject (e.g., by at least 5%, at least 10%, at least 20%, at least 30%, at least 40%, at least 50%, at least 60%, at least 70%, at least 80%, at least 90%, or at least 100%); (ii) an increase (e.g., by at least 5%, at least 10%, at least 20%, at least 30%, at least 40%, at least 50%, at least 60%, at least 70%, at least 80%, at least 90%, or at least 100%) in any of phagophore formation, autophagosome formation, lysosome numbers, lysosomal activity, LC3 puncta, lysosomal content (as determined, for example, using immunochemistry, electron microscopy, Western blotting, or flow cytometry); (iii) increased lysosome acidity (e.g., by at least 5%, at least 10%, at least 20%, at least 30%, at least 40%, at least 50%, at least 60%, at least 70%, at least 80%, at least 90%, or at least 100%); (iv) upregulation of the ubiquitin-proteasome system (e.g., by at least 5%, at least 10%, at least 20%, at least 30%, at least 40%, at least 50%, at least 60%, at least 70%, at least 80%, at least 90%, or at least 100%); or (v) inhibition of mTOR or the mTOR pathway (e.g., by at least 5%, at least 10%, at least 20%, at least 30%, at least 40%, at least 50%, at least 60%, at least 70%, at least 80%, at least 90%, or at least 100%).

### Example 6—Measuring Cell Oxidation

The success of the present composition in ameliorating cell oxidation in a subject (e.g., a human subject) can be measured, for example, by making any of the following determinations: (i) a decrease in the level of 8-isoprostane in the subject (e.g., by at least 5%, at least 10%, at least 20%, at least 30%, at least 40%, at least 50%, at least 60%, at least 70%, at least 80%, at least 90%, or at least 100%); (ii) a decrease in the level of 8-hydroxydeoxyguanosine (8-OHdG) in the subject (e.g., by at least 5%, at least 10%, at least 20%, at least 30%, at least 40%, at least 50%, at least 60%, at least 70%, at least 80%, at least 90%, or at least 100%); or (iii) a decrease in the subject of levels of any of oxidized LDL, malondialdehyde (MDA), 4-HNA, lipid hydroperoxides, oxidized proteins, protein carbonylation and protein nitration (3-nitrotyrosines), advanced glycation end products (AGEs), and advanced oxidation protein products (e.g., by at least 5%, at least 10%, at least 20%, at least 30%, at least 40%, at least 50%, at least 60%, at least 70%, at least 80%, at least 90%, or at least 100%).

### Example 7—Measuring Telomere Fitness

The success of the present composition in ameliorating telomere attrition in a subject (e.g., a human subject) can be measured, for example, by making any of the following determinations: (i) an increase in telomere length in the subject (e.g., by at least 0.01%, at least 0.1%, at least 0.5%, at least 1.0%, at least 5%, at least 10%, at least 20%, at least 30%, at least 40%, or at least 50%); or (ii) an increase in mRNA expression of telomerase, TERT, TERC, or telomerase-related genes (e.g., by at least 1%, at least 2%, at least 5%, at least 10%, at least 20%, at least 30%, at least 40%, at least 50%, at least 60%, at least 70%, at least 80%, at least 90%, or at least 100%).

### Example 8—Measuring Stem Cell Fitness

The success of the present composition in ameliorating stem cell exhaustion in a subject (e.g., a human subject) can be measured, for example, by determining an increase in the number of stem cells in vivo (e.g., by at least 1%, at least 2%, at least 5%). This increase can be measured in vitro or in vivo via, for example, one or more of the following biomarkers (i) muscle satellite stem cell biomarkers (e.g., PAX7, LMNA, and emerin); (ii) neuronal stem cell biomarkers (e.g., Nestin, Sox2, ASCL1/Mash1, ABCG2, BMI-1, beta-Catenin, Brg1, N-Cadherin, Calcitonin R, CD15/LewisX, CD133, CDCP1, COUP-TF I/NR2F1, CXCR4, FABP7/B-FABP, FABP8/M-FABP, FGFR2, FGFR4, FoxD3, Frizzled-9, GCNF/NR6A1, GFAP, Glut1, HOXB1, ID2, LRTM1, Meteorin, MSX1, Musashi-1, Musashi-2, Nestin, NeuroD1, Noggin, Notch-1, Notch-2, Nrf2, Nucleostemin, Numb, Otx2, Pax3, Pax6, PDGF R alpha, PKC zeta, Prominin 2, ROR2, RUNX1/CBFA2, RXR alpha/NR2B1 sFRP-2, SLAIN1, SOX1, SOX2, SOX9, SOX11, SOX21, SSEA-1, SSEA-4, TRAF-4, Vimentin, and ZIC1); (iii) pluripotency stem cell markers (e.g., Oct4, NANOG, Sox2, and Myc); (iv) hematopoietic stem cell markers (e.g., CD34, CD59, and CD90/Thy1); and (v) mesenchymal stem cell markers (e.g., CD105, CD90, CD73, CD44, CD45, CD29, CD166, Stro-1, CD106, and GSTT1).

Since measuring stem cells in vivo is difficult and costly, success of the present composition in ameliorating stem cell exhaustion can also be measured, for example, by making the following preferred in vitro (“lab dish”) determinations (as well as in vivo determinations): (i) an increase in the number of stem cells (e.g., by at least 2%, at least 5%, at least 10%, at least 20%, at least 30%, at least 40%, at least 50%, at least 60%, at least 70%, at least 80%, at least 90%, or at least 100%); (ii) increased viability of the stem cells (e.g., by at least 2%, at least 5%, at least 10%, at least 20%, at least 30%, at least 40%, at least 50%, at least 60%, at least 70%, at least 80%, at least 90%, or at least 100%); (iii) an increase in potency of the stem cells in vitro (and in vivo as well) (e.g., by at least 2%, at least 5%, at least 10%, at least 20%, at least 30%, at least 40%, at least 50%, at least 60%, at least 70%, at least 80%, at least 90%, or at least 100%); (iv) an increase in the quality of stem cells, measured for example by an increase in growth of the stem cells, an increase in the ability to proliferate, an increase in the ability to form colonies, or an increase in the ability to produce cells (e.g., by at least 2%, at least 5%, at least 10%, at least 20%, at least 30%, at least 40%, at least 50%, at least 60%, at least 70%, at least 80%, at least 90%, or at least 100%); (v) an increased lifespan of stem cells, as measured in hours (e.g., by at least 2%, at least 5%, at least 10%, at least 20%, at least 30%, at least 40%, at least 50%, at least 60%, at least 70%, at least 80%, at least 90%, or at least 100%); or (vi) an increased resistance of stem cells against cellular stressors (e.g., heat, cold, or toxins) (e.g., by at least 2%, at least 5%, at least 10%, at least 20%, at least 30%, at least 40%, at least 50%, at least 60%, at least 70%, at least 80%, at least 90%, or at least 100%).

The success of the present composition in ameliorating stem cell exhaustion in a subject (e.g., a human subject) can further be measured, for example, by making any of the following determinations in vitro, but also in vivo: (i) improved stem cell function as measured, for example, by replication ability, potency, proliferation capability, survival (as measured, for example, temporally (e.g., number of extra hours of survival), by exposing stem cells to physiological stressors and toxins), potency, and quality (e.g., stem cells have an increased number of replications or can generate/produce larger numbers of cells stemming from the stem cells or display increase cell division markers (e.g., cyclin D1 or increased B-catechin) (e.g., by at least 5%, at least 10%, at least 20%, at least 30%, at least 40%, at least 50%, at least 60%, at least 70%, at least 80%, at least 90%, or at least 100%); (ii) improved stem cell transcriptome (i.e., the stem cell's transcriptome is more similar to the transcriptome of a younger stem cell or a more functional stem cell) (e.g., by at least 0.5%, at least 1%, at least 2%, at least 5%, at least 10%, at least 20%, at least 30%, at least 40%, at least 50%, at least 60%, at least 70%, at least 80%, at least 90%, or at least 100%); (iii) improved stem cell proteome (i.e., the stem cell's proteome is more similar to the proteome of a younger stem cell or more functional stem cell) (e.g., by at least 0.5%, at least 1%, at least 2%, at least 5%, at least 10%, at least 20%, at least 30%, at least 40%, at least 50%, at least 60%, at least 70%, at least 80%, at least 90%, or at least 100%); or (iv) an improved stem cell epigenome (i.e., the stem cell's epigenome is more similar to the epigenome of a younger stem cell or more functional stem cell) (e.g., by at least 0.5%, at least 1%, at least 2%, at least 5%, at least 10%, at least 20%, at least 30%, at least 40%, at least 50%, at least 60%, at least 70%, at least 80%, at least 90%, or at least 100%), as assessed, for example, by measuring methylation or histonylation patterns and comparing them to those of younger, more functional, or more potent stem cells.

### Example 9—Measuring Physiological Biomarkers as Indicators of Stem Cell Fitness and Senescent Cell Levels in an Organism

The success of the present composition in ameliorating adverse physiological events in a subject (e.g., a human subject) can be measured, for example, by making any of the following determinations: (i) a decrease in the number and/or severity of skin wrinkles as measured, for example, via wrinkle surface area (e.g., via 3D topography or profilometry); (ii) an increase in skin radiance as measured, for example, via an identometer, cutometer, transepidermal water loss meter, corneometer, or a skin transcriptome or proteome biomarker panel that shows improved skin functioning; (iii) improved cognition as measured, for example, via a cognitive test; (iv) improved feelings of happiness as measured, for example, via the Depressive Symptoms Scale; (v) reduced frailty, as measured, for example, via an international frailty scale or mobility scale (such as the Canadian Study on Health & Aging Clinical Frailty Scale (CSHA-CFS)); (vi) improved reaction time; (vii) improved neuromuscular capability and strength (e.g., increased stamina, improved balance, improved proprioception, improved mobility (measured by 6-minute walk test (6MWT), 4-m gait speed test (4MGST), improved short physical performance battery (SPPB) score, or tests measuring balance, gait speed tests, chair stand tests or other markers of physical function), increased grip strength, increased power, increased activity levels, improved 400 m walk test, and improved arm curl test results); (viii) improved blood flow, as measured by increase of unit of blood per mm³ of tissue; (ix) reduced blood pressure; (x) improved vascular health, as measured, for example, via pulse wave velocity of the blood vessels or flow-mediated vasodilatation; (xi) induced weight loss, a reduction in waist circumference or BMI, or a reduction in abdominal fat volume; (xii) improvement in heart rate variability (HRV); and (xiii) improvement in an AI-assessed facial aging profile (e.g., as measured via facial AI software that for example assesses wrinkle area blood flow, or skin sagging).

Importantly, if stem cell health is improved and cell senescence is reduced, the various physiological biomarkers discussed in this example will also improve. This is particularly the case with stem cells in that reducing the aging hallmark of stem cell decline (the measurement of which is discussed above) improves physiological biomarkers.

### Example 10—Measuring Genomic Fitness

The success of the present composition in ameliorating genomic instability in a subject (e.g., a human subject) can be measured, for example, by determining a decrease in the level of 8-hydroxydeoxyguanosine (8-OHdG), gamma-H2AX, or biomarkers of DNA mutations (e.g., double strand breaks) in the subject (e.g., by at least 5%, at least 10%, at least 20%, at least 30%, at least 40%, at least 50%, at least 60%, at least 70%, at least 80%, at least 90%, or at least 100%).

### Example 11—Measuring Improvement in Deregulated Nutrient Sensing

The success of the present composition in ameliorating deregulated nutrient sensing in a subject (e.g., a human subject) can be measured, for example, by making any of the following determinations: (i) an increase in the level of insulin sensitivity in the subject (e.g., by at least 5%, at least 10%, at least 20%, at least 30%, at least 40%, at least 50%, at least 60%, at least 70%, at least 80%, at least 90%, or at least 100%); or (ii) a decrease in the level of fasting glucose, fasting insulin, LDL, HbAlc, mTOR activity, or mitochondrial reactive oxygen species in the subject (e.g., by at least 5%, at least 10%, at least 20%, at least 30%, at least 40%, at least 50%, at least 60%, at least 70%, at least 80%, at least 90%, or at least 100%).

### REFERENCES

1. The Aging Process, Denham Harman. Proceedings of the National Academy of Sciences of the United States of America, 1981.
2. Mortality in randomized trials of antioxidant supplements for primary and secondary prevention: systematic review and meta-analysis. JAMA, 2007.
3. Multivitamin-multimineral supplementation and mortality: a meta-analysis of randomized controlled trials. Am. J. Clin. Nutr., 2013.
4. Multivitamins in the prevention of cardiovascular disease in men: the Physicians' Health Study II randomized controlled trial. JAMA, 2012.
5. A Mitochondrial Superoxide Signal Triggers Increased Longevity in Caenorhabditis elegans. PLOS Biology, 2017.
6. Antioxidants prevent health-promoting effects of physical exercise in humans. Proc. Natl. Acad. Sci. USA 2009, 106, 8665-8670.
7. The Hallmarks of Aging, Cell, Vol. 153, Issue 6, pp. 1194-1217 (2013).

1. A composition comprising at least three agents selected from the group consisting of alpha-ketoglutarate, malate, pterostilbene, micro-dosed lithium, glycine, ginger, Rhodiola rosea, acetyl glucosamine, vitamin C, glucosamine, fisetin, L-theanine, oxaloacetate, fumarate, succinate, hyaluronic acid, butyrate, anthocyanins, piperlongumine, quercetin, curcuminoids, caffeine, trehalose, and spermidine.

2. The composition of claim 1, wherein the composition comprises at least three agents selected from the group consisting of alpha-ketoglutarate, malate, pterostilbene, micro-dosed lithium, glycine, ginger extract, Rhodiola rosea, acetyl glucosamine, vitamin C, glucosamine, fisetin, and L-theanine.

3. The composition of claim 1, wherein the composition comprises alpha-ketoglutarate, micro-dosed lithium, and fisetin.

4. The composition of claim 3, wherein the composition comprises alpha-ketoglutarate, micro-dosed lithium, fisetin, glycine, and pterostilbene.

5. The composition of claim 1, wherein the composition comprises alpha-ketoglutarate, micro-dosed lithium, fisetin, glycine, pterostilbene and glucosamine.

6. The composition of claim 1, wherein the composition comprises alpha-ketoglutarate, fisetin, pterostilbene, and glycine.

7. The composition of claim 6, wherein the composition comprises alpha-ketoglutarate, fisetin, glycine, pterostilbene, and glucosamine.

8. The composition of claim 1, wherein the composition comprises alpha-ketoglutarate, malate, pterostilbene, micro-dosed lithium, glycine, ginger extract, Rhodiola rosea, hyaluronic acid, vitamin C, glucosamine, fisetin, and L-theanine.

9. The composition of claim 8, wherein the composition comprises 1,100 mg of calcium alpha-ketoglutarate, 2,000 mg of magnesium malate, 50 mg of pterostilbene, 1 mg of micro-dosed lithium, 2,000 mg of glycine, 80 mg of ginger extract, 300 mg of Rhodiola rosea, 100 mg of hyaluronic acid, 100 mg of vitamin C, 1,000 mg of glucosamine sulphate, 100 mg of fisetin, and 150 mg of L-theanine.

10. The composition of claim 9, wherein the composition comprises 1,100 mg of calcium alpha-ketoglutarate, 2,000 mg of magnesium malate, 50 mg of pterostilbene, 1 mg of micro-dosed lithium, 2,000 mg of glycine, 80 mg of ginger extract, 300 mg of Rhodiola rosea, 100 mg of hyaluronic acid, 100 mg of vitamin C, 1,000 mg of glucosamine sulphate, 100 mg of fisetin, 150 mg of L-theanine, xylitol, malic acid, flavoring, silicon dioxide, calcium silicate, and rebaudioside A.

11. The composition of claim 1, wherein one or more of the agents in the composition is formulated for delayed release and/or extended release.

12. The composition of claim 11, wherein the alpha-ketoglutarate, micro-dosed lithium, fisetin, butyrate, pterostilbene, and glycine are formulated for delayed release and/or extended release.

13. The composition of claim 11, wherein each agent is formulated for delayed release and/or extended release.

14. A composition comprising (i) the composition of claim 1 and (ii) a pharmaceutically acceptable carrier.

15. A method for ameliorating at least one aging hallmark in a subject comprising administering to the subject the composition of claim 1, wherein the aging hallmark is selected from the group consisting of genomic instability, telomere attrition, an epigenetic alteration, loss of proteostasis, deregulated nutrient sensing, mitochondrial dysfunction, cellular senescence, stem cell exhaustion, and altered intercellular communication.

16. The method of claim 15, wherein the method further comprises concurrently administering a nicotinamide-based compound to the subject.

17. The method of claim 15, wherein the method ameliorates genomic instability, telomere attrition, one or more epigenetic alterations, loss of proteostasis, deregulated nutrient sensing, mitochondrial dysfunction, cellular senescence, stem cell exhaustion, and altered intercellular communication.

18. The method of claim 15, wherein the subject is a human.

19. The method of claim 15, wherein the subject is a cat, a dog, or a horse.

20. An article of manufacture comprising, in separate compartments, (i) the composition of claim 1 and (ii) a nicotinamide-based compound.

21. The article of claim 20, wherein the article comprises, in separate compartments, (i) a composition comprising alpha-ketoglutarate, malate, pterostilbene, micro-dosed lithium, glycine, ginger extract, Rhodiola rosea, hyaluronic acid, vitamin C, glucosamine, fisetin, and L-theanine, and (ii) nicotinamide mononucleotide.

22. The article of claim 21, wherein the article comprises, in separate compartments, (i) a composition comprising 1,100 mg of calcium alpha-ketoglutarate, 2,000 mg of magnesium malate, 50 mg of pterostilbene, 1 mg of micro-dosed lithium, 2,000 mg of glycine, 80 mg of ginger extract, 300 mg of Rhodiola rosea, 100 mg of hyaluronic acid, 100 mg of vitamin C, 1,000 mg of glucosamine sulphate, 100 mg of fisetin, and 150 mg of L-theanine, and (ii) 125 mg of nicotinamide mononucleotide.
